(19) United States
(12) Patent Application Publication
(10) Pub. No.: US 2013/0178538 A1
(43) Pub. Date: Jul. 11, 2013

(54) Long-Acting Injectable Analgesic Formulations for Animals

(71) Applicants: Peter Hanson, Suwanee, GA (US); Joseph K. Rosentel, JR., Johns Creek, GA (US); John Barr, Half Moon Bay, CA (US)

(72) Inventors: Peter Hanson, Suwanee, GA (US); Joseph K. Rosentel, JR., Johns Creek, GA (US); John Barr, Half Moon Bay, CA (US)

(73) Assignees: MERIAL LIMITED, Duluth, GA (US); A.P. PHARMA, Redwood City, CA (US)

(21) Appl. No.: 13/538,181

(22) Filed: Jun. 29, 2012

Related U.S. Application Data
(63) Continuation of application No. 12/505,457, filed on Jul. 17, 2009, now abandoned.
(60) Provisional application No. 61/081,561, filed on Jul. 17, 2008.

(51) Int. Cl. A61K 47/34

(52) U.S. Cl. CPC A61K 47/34
USPC 514/772.3

(57) ABSTRACT

Long acting injectable analgesic formulations and methods for providing long lasting pain relief in animals are disclosed.

### INCORPORATION BY REFERENCE

This application claims benefit of U.S. provisional application Ser. No. 61/081,561 filed Jul. 17, 2008.

All documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This application relates to formulations for providing long lasting pain relief in animals. In particular, this invention provides for improved long-acting injectable formulations for delivery of analgesics.

### BACKGROUND OF THE INVENTION

Providing analgesic effects in animals has been exhaustively studied by those of skilled in the art. By way of example, the U.S. Department of Agriculture Animal Welfare Information Center (AWIC) published a compilation of reference entitled “A Reference Source for Analgesia & Analgesics in Animals” which discloses over 900 references as they relate to the use of analgesia and analgesics generally and in amphibians/reptiles, avians (birds), bovines, dogs and cats, equines, ferrets, fish/crabs/snails/shrimp/mollusks/shell fish, goats, guinea pigs, mice, marine mammals, non-domestic/wild/exotic animals, primates, rabbits and rodents, rats, sheep and swine (each of these references are also intended to be incorporated by reference).

Analgesics are administered by a variety of routes including, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The particular route of administration selected by the practitioner depends upon factors such as the physiochemical properties of the pharmaceutical or therapeutic agent, the condition of the host, and economic factors.

A general problem with regard to the delivery of analgesics is trying to maintain a steady concentration of the analgesic and also the relatively short term effectiveness of the analgesic formulations in use which requires multiple administrations of analgesic dosages (e.g. 3-4 hours or 6-8 hours). Exacerbating the problem is the fact that some analgesics have the risk of addiction (e.g. heroin), and are classified as narcotics under the Controlled Substances Act, and/or induce nausea and other undesirable side effects.

Solutions to this problem include the use of patient-controlled analgesia (PCA) or computer-assisted continuous infusion (CACI). However, for veterinary or livestock animals it is impractical to allow the “patient” control of the dosing of the analgesic or to continuously titrate the amount of intravenous agent present in each animal. Transdermal patches, as with fentanyl, have been transferred from human to extra-label usage on animals. Limitations exist with availability of non-haired skin for patch application, animals removing or eating the patches, and the potential dispensing of controlled substances for use outside a clinic setting. These solutions also do not allow for increasing the dosage of analgesic to be administered initially in order to achieve a long lasting effect. Therefore, there is still a need in the art for providing a slow release of therapeutic agent and which thereby provides sustained concentration of an analgesic and long acting effects to an animal.

Oral formulations are a convenient means of delivering an active agent but have the problem of “bioavailability”, which indicates the percentage of a drug dose which reaches its site of action, or a biological fluid, from which the drug has access, to its site of action (Goodman & Gilman's The Pharmacological Basis of Therapeutics, Hardman, J. G., Limbird, L. E., and Gilman, A. G., eds., Tenth Ed., McGraw-Hill, 2001). The bioavailability of drugs is a complex issue. For example, a drug given orally must be absorbed first from the stomach and intestine, but this may be limited by the characteristics of the dosage form and/or the drug's physicochemical properties. In addition drug then passes through the liver, where metabolism and/or biliary excretion may occur before it reaches the systemic circulation. Accordingly, a fraction of the administered and absorbed dose of drug will be inactivated or diverted before it can reach the general circulation and be distributed to its sites of action. If the metabolic or excretory capacity of the liver for the agent in question is large, bioavailability will be substantially reduced (the so-called first pass effect). This decrease in availability is a function of the anatomical site from which absorption takes place; other anatomical, physiological, and pathological factors can influence bioavailability and the choice of the route of administration must be based on an understanding of these conditions.

One obvious way to change the bioavailability of a therapeutic agent is to change the route of administration from, for example, oral to parenteral. However, the use of parenteral injection may not always be appropriate. For example, intravenous injection has an increased risk of adverse effects and is not suitable for oily solutions or insoluble substances. Subcutaneous injections are not suitable for large volumes and may present possible pain or necrosis from irritating substances. Other strategies include increasing drug potency, changing dosage regimens, or using combination therapies. Furthermore, the choice of pharmaceutical formulation plays a role in rendering the therapeutic agent effective upon administration.

Analgesic usage in veterinary medicine presents other unique considerations. Animal patients vary from small companion animals and birds that live in intimate proximity to their owners to pastured food and fiber producing animals with little human contact. The animal species, their human contact, temperament, size, use, emotional and economic value, and pathological conditions are all important factors that must be considered in selecting an appropriate type of analgesic and administration route for therapy.

The particular dosage form varies based upon the kind of analgesic used, the animal species being treated, and on whether the type of pain is suitable for treatment via local or systemic delivery. Local analgesic therapy achieves a high concentration of analgesic at the source of the pain (e.g., within a joint), thus potentially avoiding the adverse effects that are associated with systemic analgesic therapy. Where the source of the pain is in multiple locations or multisystemic, then parenteral or systemic delivery is desired.

Parenteral administration of analgesics is often preferred as a treatment mode for food animals. Therefore, analgesic treatment of pastured animals or large companion animals generally requires confinement of these animals for the duration of therapy. However, repeated restraint and administration within a relatively short period of time add to the stress of illness and may complicate convalescence and recovery. Even docile animals tend to become fractious and uncooperative after multiple days of parenteral therapy.

It is therefore evident from the foregoing description that there are advantages of systemic and local delivery of long-acting analgesic formulations to food producing and companion animals, and birds for providing pain relief. Some of these advantages include improved patient compliance, convenience for the owner and veterinarians, and improved cost effectiveness of providing pain relief. Long-acting analgesic formulations can even reduce the amount of analgesics used for therapy in animals, since the convenient and easily administered long-acting formulations make it possible to treat each affected animal in a more efficient and effective manner.

Several different approaches to develop long-acting analgesic formulations have been explored. These include formulating injectable formulations such as suspensions, concentrated solutions, injectable gels and microparticles and implants. The selection of the development approach of long-acting analgesic formulations is determined by the intended application criteria, such as type of disease, systemic or local therapy, short-term or long-term therapy and type of animals being treated.

Biodegradable polymers have been used in parenteral controlled release formulations of bioactive compounds. Gels prepared with biodegradable polymers such as poly(lactide-co-glycolide), poly(lactic acid) and polyoxyethylene polyoxypropylene block copolymers (poloxamers or, LUTROL® F) and biocompatible, non-toxic solvents, such as triethyl citrate and acetyl triethyl citrate or water have been used to develop long-acting analgesics formulations. The reversible thermal gelation characteristics of the formulations allowed the liquid injection to gel at the injection site at body temperature.

In one approach the polymer is fabricated into microspheres that may be injected via syringe, and the bioactive compound is entrapped within the microspheres. This approach has certain challenges in part due to the difficulty in the manufacturing procedure for producing sterile and reproducible products, and the high cost of manufacturing. In another approach the biodegradable polymer and the bioactive material are dissolved in a biocompatible water-miscible solvent to provide a liquid composition. When the liquid composition is injected into the body, the solvent dissipates into the surrounding aqueous environment, and the polymer forms a solid depot from which the bioactive material is released.

U.S. Pat. No. 4,938,763, which is incorporated herein by reference in its entirety, concerns polymeric compositions having a thermoplastic polymer, rate modifying agent, water soluble bioactive material and water-miscible organic solvent. Upon exposure to an aqueous environment (e.g. body fluids) the liquid composition is capable of forming a biodegradable microporous, solid polymer matrix for controlled release of water soluble or dispersible bioactive materials over about four weeks. The thermoplastic polymer may be, among many listed, polylactide, polyglycolide, polycaprolactone or copolymers thereof, and is used in high concentration (45 to 50%). The rate modifying agent may be, among many others listed, glycerol triacetate (triacetin); however, only ethyl heptanoate is exemplified; and the amount of the rate modifying agent is no more than 15%.

Indeed, with respect to the patent literature, reference is made to: U.S. Pat. Nos. 4,150,108, 4,329,332, 4,331,652, 4,333,919, 4,389,330, 4,489,055, 4,526,938, 4,530,840, 4,542,025, 4,563,489, 4,675,189, 4,677,191, 4,683,288, 4,758,435, 5,599,852, 5,607,686, 5,609,886, 5,631,015, 5,654,010, 5,700,485, 5,702,717, 5,711,968, 5,733,566, 4,938,763, 5,077,049, 5,278,201, 5,278,202, 5,288,496, 5,324,519, 5,324,520, 5,340,849, 5,368,859, 5,401,507, 5,419,910, 5,427,796, 5,487,897, 5,599,552, 5,632,727, 5,643,595, 5,660,849, 5,686,092, 5,702,716, 5,707,647, 5,725,491, 5,733,950, 5,736,152, 5,744,153, 5,759,563, and 5,780,044, all of which are incorporated herein by reference in their entirety. These documents tend to provide compositions that form a solid, gel or coagulated mass; for instance, a significant amount of polymer is contemplated in these documents, akin to U.S. Pat. No. 4,938,763, which is incorporated herein by reference in its entirety.

Mention is also made of: Shah et al (J. Controlled Release, 1993, 27:139-147), as relating to formulations for sustained release of bioactive compounds containing various concentrations of poly(lactic-co-glycolic) acid copolymer (PLGA) dissolved in vehicles such as triacetin; Lambert and Peck (J. Controlled Release, 1995, 33:189-195), as a study of the release of protein from a 20% PLGA solution in N-methylpyrrolidone exposed to aqueous fluid; and Shivley et al (J. Controlled Release, 1995, 33:237-243), as a study of the solubility parameter of poly(lactide-co-glycolide) copolymer in a variety of solvents, and the in vivo release of naltrexone from two injectable implants (5% naltrexone in either 57% PLGA and 38% N-methylpyrrolidone or 35% PLGA and 60% N-methylpyrrolidone).

Although most of the analgesics currently on the market can generally be used in any animal species, developing a long-acting formulation which is suitable requires consideration of the size of animal species, physiological features of the animal, diseases to be treated, and the economic and emotional interest of the animal owners.

With all of the above factors at play in the development of analgesic formulations, it remains a challenge to develop long-acting injectable formulations that have long lasting effects in order that a single injection is all that is necessary. Surprisingly, the injectable formulation of the present invention addresses the problems associated with analgesic delivery and fulfills this long-felt need in the art.

Citation or identification of any document in this application is not an admission that such document is available as prior art to the present invention.

### SUMMARY OF THE INVENTION

The present invention relates to novel long-acting injectable (LAI) formulations that provide slow release of therapeutic agent and which thereby provide sustained concentrations of therapeutic agent, which provides long lasting pain relief Such a dosage regimen allows for convenience in administration, increases in compliance, and decreases in error in treatment.

In a first aspect of the invention, the LAI formulation comprises an analgesic, a polyorthoester and optionally, a pharmaceutically acceptable excipient or carrier.

In a second aspect of the invention, the LAI formulation of the first aspect of the invention is prepared by mixing the analgesic with the polyorthoester and a pharmaceutically acceptable excipient.

A third aspect of the invention is directed toward the systemic administration of the LAI formulation of the first aspect of the invention to provide long acting analgesic effect and thereby effectively providing long lasting pain relief in an animal with a single administration.

A fourth aspect of the invention is directed toward the local administration of the LAI formulation of the present invention to provide, by a single injection, slow release of analgesic and sustained concentrations of therapeutic agent, for long acting effect, and thereby effectively providing long lasting pain relief in an animal with a single injection.

These and other embodiments are disclosed or are obvious from and encompassed by, the following Detailed Description.

### BRIEF DESCRIPTION OF DRAWINGS

The following detailed description, given by way of example, and which is not intended to limit the invention to specific embodiments described, may be understood in conjunction with the accompanying figures, incorporated herein by reference, in which:

FIG. 1 shows the plasma levels when long-acting buprenorphine compositions containing 0.5%, 1% and 2% w/w (buprenorphine/polyorthoester) were administered to dogs at a dosage rate of 0.1 mg/kg.

FIG. 2 shows the plasma levels when long-acting buprenorphine composition containing 2% w/w (buprenorphine/polyorthoester) was administered to dogs at various dosage rates (0.11, 0.22, and 0.34 mg/kg).

FIG. 3 shows the plasma levels when long-acting buprenorphine composition containing 0.5% w/w (buprenorphine/polyorthoester) was administered to dogs at various dosage rates (0.025, 0.05, and 0.075 mg/kg).

FIG. 4 shows the thermal threshold for the compositions of the invention in a thermal stimulation model at three different dosage levels (0.025 mg/kg, 0.05 mg/kg and 0.075 mg/kg). The 0.075 mg/kg dosage level is shown to be efficacious for at least 48 hours at the 99% confidence level.

FIG. 5 shows the thermal threshold for the commercially available TEMGESIC control in a thermal stimulation model up to 6 hours (typically 4-6 hour clinical redosing period).

FIG. 6 shows the efficacy of the compositions of the invention in an electrical stimuli model at three different dosage levels (0.025 mg/kg, 0.05 mg/kg and 0.075 mg/kg). Values above 30% change from baseline are considered clinically efficacious.

FIG. 7 shows the efficacy of the TEMGESIC control in an electrical stimuli model, showing analgesic efficacy only up to 4 hours post dosing.

### DETAILED DESCRIPTION

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

The term “clearance” as used herein refers to the removal of a substance from the blood, e.g., by renal excretion, expressed in terms of the volume flow of blood or plasma that would contain the amount of substance removed per unit time.

The term “half-life” as used herein refers to the period of time required for one-half of an amount of a substance to be lost through biological processes.

The term “bioavailability” as used herein refers to the physiological availability of a given amount of a drug, as distinct from its chemical potency. The term may also refer to the proportion of the administered dose which is absorbed into the bloodstream.

The term “animal” is used herein to include all mammals, birds and fish. The animal as used herein may be selected from the group consisting of equine (e.g., horse), canine (e.g., dogs, wolves, foxes, coyotes, jackals), feline (e.g., lions, tigers, domestic cats, wild cats, other big cats, and other felines including cheetahs and lynx), bovine (e.g., cattle), porcine (e.g., pig), avian (e.g., chicken, duck, goose, turkey, quail, pheasant, parrot, finches, hawk, crow, ostrich, emu and cassowary), primate (e.g., prosimian, tarsier, monkey, gibbon, ape), humans, and fish. The term “animal” also includes an individual animal in all stages of development, including embryonic and fetal stages.

The term “long acting” or “long lasting” as used herein refers to a period of time of at least about 12 hours to about 30 days. All possible ranges within this range are also considered to be part of the invention (e.g., about 12 hours to about 48 hours; about 24 hours to about 72 hours; about 3 days to about 5 days; about 5 days to about 7 days; about 7 days to about 10 days)

The present invention provides for a long-acting injectable (LAI) formulation, which comprises at least one analgesic, at least one polyorthoester, and at least one aqueous solvent.

The analgesic may be selected from the following, which is to be considered non-limiting opioid agonists, opioid antagonists, non-opioid analgesics and combinations thereof.

In one aspect of the invention, the opioid analgesic is an opioid agonist, an opioid antagonist or a combination thereof.

In one embodiment of the opioid analgesic, the opioid agonist includes but is not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, remifentanil, sufentanil, tilidine, tramadol, pharmaceutically acceptable salts thereof, and mixtures thereof.

In another embodiment of the opioid analgesic, the opioid agonist is buprenorphine or a pharmaceutically acceptable salt thereof.

In yet another embodiment of the opioid analgesic, the opioid antagonist includes but is not limited to naloxone (U.S. Pat. No. 3,254,088, which is incorporated herein by reference in its entirety), naltrexone (U.S. Pat. No. 3,332,950, which is incorporated herein by reference in its entirety) and mixtures thereof; or a pharmaceutically acceptable salt thereof.

In still another embodiment of the opioid analgesic, the analgesic is a combination of an opioid agonist and opioid antagonist (examples include, but are not limited to, suboxone which is a combination of buprenorphine and naloxone).

In another aspect of the invention, the opioid analgesic is combined with a non-opioid analgesic.

In one embodiment, the non-opioid analgesic includes, but is not limited to, non-steroidal anti-inflammatory drugs (NSAIDs), such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamnic acid, meclofenamic acid, flufenamic acid, niflumic acid, tolfenamic acid, diflurisal, flufenisal, piroxicam, sudoxicam, isoxicam, and pharmaceutically acceptable salts thereof, and mixtures thereof.

In another embodiment, the non-opioid analgesics include the following non-limiting chemical classes of analgesic, antipyretic, non-steroidal anti-inflammatory drugs (NSAIDs): salicylic acid derivatives, including sodium salicylate, choline magnesium trisalicylate, salsalate, diflunisal, salicylsalicylic acid, sulfasalazine, and olsalazine; para-aminophenol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid and meclofenamic acid; enolic acids, including oxicams (piroxicam, tenoxicam), and pyrazolidinediones (phenylbutazone, oxyphenthartazone); and alkanones, including nabumetone.

For a more detailed description of the analgesics, see “Chapter 23—Opioid Analgesics” by Gutstein et al. (pages 569-619) and “Chapter 27—Analgesic-Antipyretic and Antiinflammatory Agents and Drugs Employed in the Treatment of Gout” by Roberts et al. (pages 687-731), both from Goodman & Gilman's The Pharmacological Basis of Therapeutics, Joel G. Hardman and Lee E. Limbird, eds., 10^{th} Ed., pages 569-619, (2001)) and Glen R. Hanson, “Analgesic, Antipyretic and Anti-Inflammatory Drugs” in Remington: The Science and Practice of Pharmacy, A. R. Gennaro ed. 19th ed., vol. II: 1196-1221 (1995).

In still another embodiment, the non-opioid analgesic include COX-2 inhibitors and 5-lipoxygenase inhibitors, as well as combinations thereof, as described in U.S. Pat. No. 6,136,839, which is incorporated herein by reference in its entirety. Examples of useful COX-2 inhibitors include, but are not limited to, rofecoxib, celecoxib, deracoxib, and firocoxib.

In still another embodiment, the non-opioid analgesic include tachykinin antagonists as described in U.S. Pat. No. 6,180,624, which is incorporated herein by reference in its entirety, and NMDA (N-methyl-D-aspartate) NR2B subtype antagonists as described in U.S. Pat. No. 6,538,008, which is incorporated herein by reference in its entirety.

In one embodiment, the LAI formulations contain about 0.01 to about 50% by weight (w/w) of an analgesic. In another embodiment of the invention, the LAI formulations contain about 0.1 to about 10% by weight (w/w) of an analgesic. In another embodiment of the invention, the LAI formulations contain about 0.1 to about 5% by weight (w/w) of an analgesic. In yet another embodiment of the invention, the LAI formulations contain about 0.1 to about 2% by weight (w/w) of an analgesic. In still another embodiment of the invention, the LAI formulations contain about 0.25 to about 0.75% by weight (w/w) of an analgesic.

In another embodiment, the long-acting injectable formulation contains buprenorphine, or salts thereof. In one embodiment of the invention, the buprenorphine is present in an amount of about 1%-about 2% by weight in the formulation.

The polyorthoester may be selected from the polyorthoesters described in U.S. Pat. Nos. 6,524,606; 6,590,059; 6,613,355; 6,667,371; 6,790,458; 6,822,000; 6,861,068; 6,863,782; 6,946,145; 7,045,589; and 7,163,694, all of which are incorporated by reference herein in their entirety.

Processes for the preparation of orthoesters are well known in the art. The orthoesters described herein may be prepared by any processes known in the art. In one embodiment of the invention, the polyorthoester is prepared by a reaction between 3,9-di(ethylidene)-2,4,8,10-tetraoxaspiro[5.5]undecane (DETOSU), triethylene glycol (TEG) and triethyleneglycol monoglycolide (TEG-mGL).

In another embodiment, the polyorthoester is prepared by a condensation reaction between one or more diols and a diketene acetal.

The polyorthoesters include but are not limited to the polyorthoesters described in U.S. Pat. No. 7,045,589, which is incorporated herein by reference in its entirety, which include but are not limited to the compounds of formula (I):

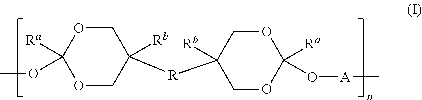

wherein:
n is an integer of at least 5;
R is a bond, —(CH₂)ₐ—, or —(CH₂)_{b}—O—(CH₂)_{c}—, where a is an integer of 1 to 10, and b and c are independently integers of 1 to 5;
R^{a} is a C₁-C₄ alkyl;
R^{b} is a C₁-C₄ alkyl; and
each A is independently selected from R¹, R², R³, and R⁴, where
R¹ is:

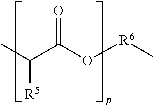

wherein:
p is an integer of 1 to 20;
R⁵ is hydrogen or C₁-C₄ alkyl; and
R⁶ is:

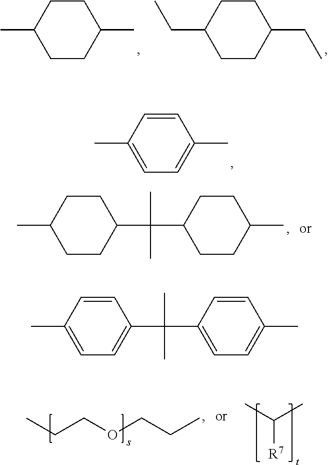

wherein:
   s is an integer of 0 to 30;
   t is an integer of 2 to 200; and
   R⁷ is hydrogen or C₁-C₄ alkyl;
R² is:

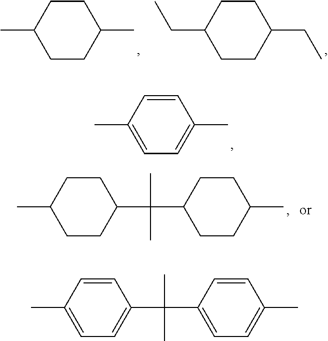

R³ is:

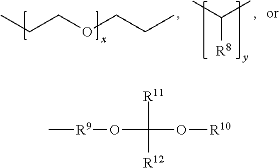

where:
   x is an integer of 0 to 30;
   y is an integer of 2 to 200;
   R⁸ is hydrogen or C₁-C₄ alkyl;
   R⁹ and R¹⁰ are independently C₁-C₁₂ alkylene;
   R¹¹ is hydrogen or C₁-C₆ alkyl and R¹² is C₁-C₆ alkyl;
   or R¹¹ and R¹² together are C₃-C₁₀ alkylene; and
R⁴ is:
(i) the residue of a diol containing at least one amine functionality incorporated therein, or
(ii) the residue of a diol containing at least one functional group independently selected from amide, imide, urea, and urethane groups.

The polyorthoesters include but are not limited to the polyorthoesters described in U.S. Pat. No. 6,790,458, which is incorporated herein by reference in its entirety, which include but are not limited to the compounds of formula (II):

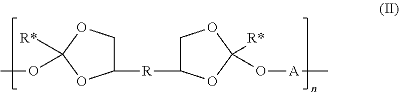

wherein:
R is a bond, —(CH₂)ₐ—, or —(CH₂)_{b}—O—(CH₂)_{c}—; where a is an integer of 1 to 10, and b and c are independently integers of 1 to 5;
R* is a C₁-C₄ alkyl;
n is an integer of at least 5; and
A is R¹, R², R³, or R⁴, where
R¹ is:

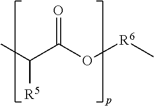

wherein:
p is an integer of 1 to 20;
R⁵ is hydrogen or C₁-C₄ alkyl; and
R⁶ is

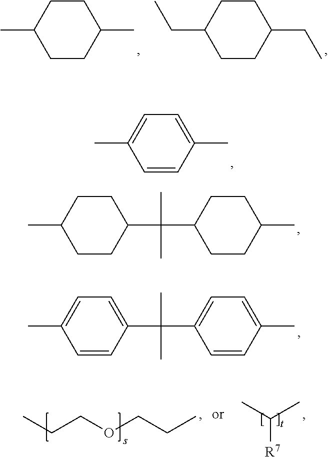

wherein:
   s is an integer of 0 to 30;
   t is an integer of 2 to 200; and
   R⁷ is hydrogen or C₁-C₄ alkyl;
R² is:

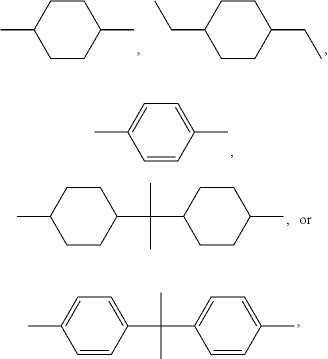

R³ is:

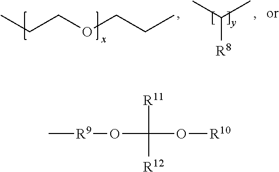

wherein:
x is an integer of 0 to 30;
y is an integer of 2 to 200; and
R⁸ is hydrogen or C₁-C₄ alkyl;
R⁹ and R¹⁰ are independently C₁-C₁₂ alkylene;
R¹¹ is hydrogen or C₁-C₆ alkyl and R¹² is C₁-C₆ alkyl;
or R¹¹ and R¹² together are C₃-C₁₀ alkylene; and
R⁴ is a diol containing at least one functional group independently selected from amide, imide, urea and urethane groups;
in which at least 0.1 mol percent of the A units are of the formula R¹.

The polyorthoesters include but are not limited to the polyorthoesters described in U.S. Pat. No. 5,968,543, which is incorporated herein by reference in its entirety, which include but are not limited to the compounds of formula:

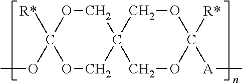

where R* is a C₁-C₄ alkyl;
each A is selected from the group consisting of —O—R¹—, —O—R²—, or (—O—R³)_{q}—, where q is 1 to 20;
n is at least 5; and
R¹ is

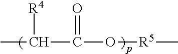

in which
p is 1-10;
R⁴ is hydrogen or a C₁-C₆ alkyl; and
R⁵ is

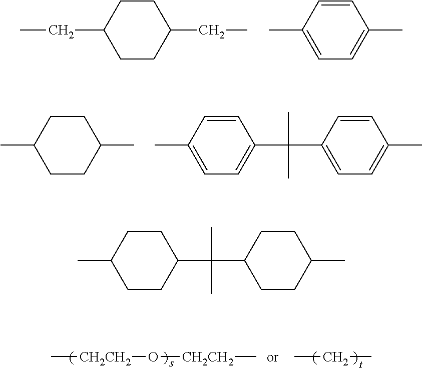

where:
s is 1 to 100;
t is 1 to 12;
R² is

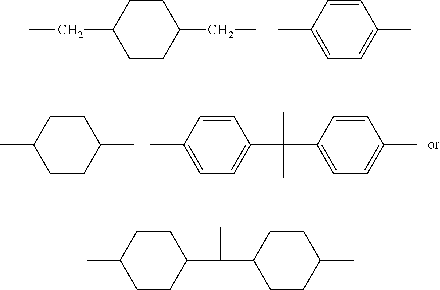

when q is 1, R³ is

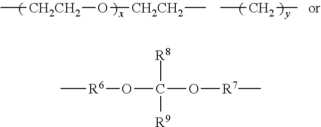

in which:
x is 1 to 100;
y is 1 to 12;
R⁶ and R⁷ are independently a C₁-C₁₂ alkylene;
R⁸ is hydrogen or a C₁-C₆ alkyl; and
R⁹ is a C₁-C₆ alkyl; or
R⁸ and R⁹ taken together are a C₃-C₁₀ alkylene; and
   when q is 2 to 20, each R³ may be the same or different and is

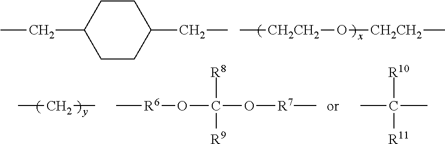

where x, y, R⁶, R⁷, R⁸ and R⁹ are defined above,
R¹⁰ is hydrogen or a C₁-C₄ alkyl,
and R¹¹ is a C₁-C₄ alkyl; provided that the polymer comprises at least 0.1 mole percent of units in which A is —O—R¹—.

Specific polyorthoesters which may be used in the invention include but are not limited to APF 579, APF 579R, APF 580, APF 580R, APF 626 and APF 626R (products from A.P. Pharma, Redwood City, Calif.).

In one embodiment, polyorthoesters of the present invention are treated under various conditions to enhance the stability when stored at room temperature and to improve the time-controlled release of the active pharmaceutical agent in the host animal. The conditions comprise one or a combination of two or more of the factors: an elevated temperature, an inert gas, reduced oxygen concentration, reduced humidity, elevated or reduced pressure, elevated or reduced mixing speed, a sufficient treatment time, and a mixture thereof. The elevated temperature contemplated in the present invention includes, but is not limited to, at least about 60° C., at least about 65° C., at least about 70° C., at least about 75° C., at least about 80° C., at least about 85° C., at least about 90° C., from about 60° C. to about 130° C., or from about 70° C. to 120° C. In another embodiment, the inert gas is argon. In yet another embodiment, the treatment time is from about 10 minutes to about 30 hours. In yet another embodiment, the treatment time is about 15 minutes, about 30 minutes, about 1 hour, about 2 hours, about 3 hours, about 4 hours, about 5 hours, from about 5 hours to about 10 hours, from about 10 hours to about 15 hours, from about 15 hours to about 20 hours, from about 20 hours to about 24 hours, from about 24 hours to about 30 hours.

The polyorthoester may be selected from the polyorthoesters described in U.S. provisional application No. 61/121,894 filed on Dec. 11, 2008, the entire content of which is incorporated herein by reference.

The excipients suitable for the present invention are pharmaceutically acceptable and polyorthoester-compatible materials. They are liquid at room temperature, and are readily miscible with the polyorthoesters.

Suitable excipients include, but are not limited to, poly(ethylene glycol) ether derivatives having a molecular weight of between 200 and 4,000, such as poly(ethylene glycol) mono- or di-alkyl ethers, preferably poly(ethylene glycol)monomethyl ether 550 or poly(ethylene glycol)dimethyl ether 250; poly(ethylene glycol) copolymers having a molecular weight of between 400 and 4,000 such as poly(ethylene glycol-co-polypropylene glycol); propylene glycol mono- or di-esters of a C₂₋₁₉ aliphatic carboxylic acid or a mixture of such acids, such as propylene glycol dicaprylate or dicaprate; mono-, di- or tri-glycerides of a C₂₋₁₉ aliphatic carboxylic acid or a mixture of such acids, such as glyceryl caprylate, glyceryl caprate, glyceryl caprylate/caprate, glyceryl caprylate/caprate/laurate, glycofurol and similar ethoxylated tetrahydrofurfuryl alcohols and their C₁₋₄ alkyl ethers and C₂₋₁₉, aliphatic carboxylic acid esters; and biocompatible oils such as sunflower oil, sesame oil and other non- or partially-hydrogenated vegetable oils.

Most of these materials are commercially available, for example, from Aldrich Chemical Company (Milwaukee, Wis.), Abitec Corporation (Columbus, Ohio), LIPO Chemicals Inc. (Paterson, N.J.), Dow Chemical Company (Plaquemine, La.), and Jarchem Industries, Inc. (Newark, N.J.).

The concentration of the polyorthoester may be in the range of 1-99 wt. %, 5-40 wt. %, 5-30 wt. %, 10-30 wt. %, 5-20 wt. % or 10-20 wt. % of the composition. The total concentration of the excipient may be 1-90 wt. %, 5-60 wt. %, or 10-50 wt. %, of the composition.

Other additives or ingredients known in the art can be added to the LAI formulation of the invention (see e.g. Plumb' Veterinary Drug Handbook, 5^{th} Edition, ed. Donald C. Plumb, Blackwell Publishing, (2005) or The Merck Veterinary Manual, 9^{th} Edition, (January 2005)).

The long-acting injectable formulation of the invention may be prepared by adding the therapeutic agent with a polyorthoester and mixing until uniform. Optionally, a pharmaceutically acceptable excipient can be added during or after the mixing step. Since the long acting formulation is intended for injection, it is necessary that it be sterilized. As the formulation is generally too viscous for membrane filtration, sterilization via gamma irradiation or E-beam irradiation is used for the formulations of the invention.

The long-acting injectable formulation of the invention may be prepared by adding the analgesic with the polyorthoester and mixing until uniform. Since these formulations are less viscous, membrane sterilization is preferred. The sterile mixture is further mixed with sterile water for injection, q.s. to 100%.

The inventive formulations herein described may be used to treat pain in animals, including humans, caused by various conditions including a number of disease states, by administering an effective amount of the formulations of the invention to the animal in need thereof. The determining of a treatment protocol of a specific indication would be well within the skill level of a practitioner in the pharmaceutical or veterinary arts.

The inventive formulations herein described may be administered to a warm-blooded animals, such as cattle, sheep, goats, pigs, cats, dogs, horses, llamas, deer, rabbits, skunks, raccoons, primates, humans, camels and the like, or birds. The amount of pharmaceutical active agent depends on the individual therapeutic agent, the animal being treated, the disease state, and the severity of the disease state. The determination of those factors is well within the skill level of the practitioner.

In one embodiment of the invention, the LAI formulation of the invention is administered parenterally to an animal in need thereof in order to provide long lasting pain relief. In another embodiment of the invention, the long lasting pain relief is for a period of time including, but not limited to, about 2 to about 48 hours, about 2 to about 12 hours, about 2 to about 6 hours, about 6 hours to about 12 hours, 6 hours to about 48 hours, 6 hours to about 24 hours, 12 hours to about 48 hours, about 24 hours to about 72 hours, about 3 to 5 days, about 5 days to about 7 days, and about 7 days to about 10 days.

In another embodiment for the administration of the LAI formulation of the invention, the amount of analgesic delivered to the animal in a single dose can be higher than the recommended or guideline dosage of an analgesic administered in a typical form because of the controlled release mechanism of the LAI formulation will provide controlled safe release of the active so that overdose is not a concern. The higher dosage can be in a range of about 5 to about 50 times higher, about 10 to about 25 times higher or about 12.5 to about 20 times higher than the recommended dosage.

By way of example, a suggested dosage from Plumb's Veterinary Drug Handbook for pain management using buprenorphine is 0.005-0.2 mg/kg (mg of buprenorphine per kg of weight of the patient) intramuscularly (IM), or intravenously (IV) or subcutaneous (SC) for dogs every 6-12 hours, 0.005-0.01 mg/kg IM, IV or SC for cats every 6-12 hours. Surprisingly, buprenorphine delivered in the LAI of the invention can be administered at a dosage rate ranges of about 0.01-about 1.0 mg/kg; about 0.025-about 0.5 mg/kg; about 0.75-about 0.4 mg/kg.; and about 0.1 mg/kg-about 0.4 mg/kg; providing pain relief for about 12 hours to about 10 days.

The invention is further described by the following non-limiting examples which further illustrate the invention, and are not intended, nor should they be interpreted to, limit the scope of the invention.

### EXAMPLES

### Analysis of Plasma Concentration of Analgesics

A bioanalytical method for the determination of an analgesic from canine, feline or other animal serum samples was developed using Reversed-Phase HPLC with UV Detection. All serum samples were extracted using a liquid-liquid extraction procedure and injected on an HPLC with UV absorption at 210 nm. Sets of fortified control samples to assess method performance, along with an unfortified control sample were included to assess any inherent interference.

Pharmacokinetic analysis was performed using WinNonlin software, version 4.0 (Pharsight Corporation, Mountain View, Calif., 2002). The area under the plasma concentration-time curve (AUC) was calculated using the linear/logarithmic trapezoidal method from 0 to the last point at which drug concentration was quantified [AUC(0-tₗₐₛₜ)]. Clearance and volume of distribution values, not corrected for bioavailability, were also calculated for each animal. The terminal elimination half life was calculated via linear regression of the last two to four nonzero values. Cₘₐₓ and Tₘₐₓ for each animal were taken as the highest observed concentration and time to that observation.

### Assessment of Analgesic Efficacy

The efficacy of the inventive formulations for the relief of pain in animals may be assessed by different models known in the art. Representative methods for the assessment of analgesic efficacy in cats and dogs are described below. It will be apparent to those of skill in the art that other known models for testing analgesic efficacy in other animals, including humans, may be used.

1. Thermal stimulation model in cats and dogs: The analgesic efficacy of the formulations of the invention in cats and dogs may be measured by testing the thermal threshold of cats and dogs treated with the inventive formulations compared to control animals (see Steagall et al., Veterinary Anaesthesia and Analgesia, 2007, 34, 344-350; Robertson et al., The Veterinary Record, Oct. 11, 2003, 462-465). The thermal thresholds are measured by applying a mild, transient heat stimulus to elicit pain. When activated, the probe is heated at 0.6° C. per second with an automatic cut off at 55° C. The heater is activated and then switched off as soon as the cat or dog reacts to the heat generated, typically by some physical movement, or by vocalization. At the point of reaction, the probe temperature is recorded as the thermal threshold. Prior to drug administration, four measurements are taken at 15 minute intervals, and their mean value is taken as the control thermal threshold.

Alternatively, thermal stimulation in cats may be administered by pointing a laser of sufficient power to a paw of test animals through a transparent platform on which the test animals are seated. The time required to elicit a response to the thermal stimulus is recorded. Prior to drug administration, four measurements are taken at 15 minute intervals and the mean value of the time required to elicit a response to the stimulus is taken as the control thermal threshold.

2. Nociceptive Withdrawal Reflex (NWR) in dogs: The analgesic efficacy of the inventive formulations may be tested according to the procedure reported by Bergadano et al. (see Bergadano et al., Am J Vet Res., vol. 68(8), August 2007; Am J Vet Res., vol. 67(5), May 2006): Briefly, the sites for stimulation and recording are clipped, shaved and degreased. The dog is placed in right lateral recumbency in a comfortable, commercial dog bed. The limbs are extended laterally in a natural position, but not supported and without weight bearing or movement restriction of the nondependent limb. The surface electrodes are then positioned, the nerves are transcutaneously stimulated by electrical stimuli, and the response is recorded by surface electromyography (EMG). Dogs initially receive 4 test stimuli at different intensities to familiarize them with the method prior to threshold measurement. The initial current intensity delivered is 1 mA. If no reflex is elicited, the current is gradually increased in steps of 0.2 mA until an EMG response is evoked. The threshold current intensity required to elicit an EMG response of dogs treated with the inventive formulations compared to control animals is determined.
3. Clinical Study: The analgesic efficacy in cats and dogs may also be tested in a clinical setting. Laboratory or client-owned cats and dogs are utilized/enrolled based on need for soft-tissue or orthopedic surgery. The analgesic efficacy of the formulations is measured on a validated behavior pain scale, such as short form of the Glasgow Composite Measure Pain Scale for a set period of time (e.g. 3-5 days post-operation; see Reid, J. et al. Vet. Anaesth. Analg. 2005; 25:1-7).

### Example 1

### Preparation of Buprenorphine/Polyorthoester (APF580R) Formulation

This example provides a procedure in which the formulation was treated to enhance the stability at room temperature and to improve the controlled release of the active pharmaceutical agent in the host animal.

TABLE 1
Composition of APF580R* containing 2% w/w buprenorphine

| | Quantity | |
|---|---|---|
| Component | (% w/w) | Function |
| AP135 | 78.4 | Excipient |
| MPEG-550 | 19.6 | Excipient |
| Buprenorphine | 2 | Active Pharmaceutical |
| | | Ingredient |

*The “R” designation refers to “relaxed” which is a controlled reduction in molecular weight and viscosity of the TEG-polyorthoester polymer vehicle using thermal stress in a low water and inert gas environment.

To prepare APF580R containing 2% w/w buprenorphine, appropriate amounts of raw materials were weighed. AP135 polymer (Sigma Aldrich Fine Chemicals, Madison, Wis.) was warmed at about 70° C. under Argon. Buprenorphine base was dissolved in MPEG-550 (polyethylene glycol monomethyl ether, number average molecular weight 550 Daltons, Dow Chemical Company, Plaquemine, La.) at around 120° C. for about 15 minutes under Argon. The AP135 polymer was then mixed with the buprenorphine/MPEG-550 solution at about 70° C. for about 30 minutes. The mixture was heated to about 90° C. and kept at 90° C. for about 24 hours. The bulk drug product was then packaged into individual syringes under Argon and the filled syringes were sterilized by gamma irradiation.

AP135 is a triethylene glycol based polyorthoester commonly referred to by the acronym TEG-POE. AP135 is the compositional code number assigned to this copolymer. Starting materials and AP135 are manufactured by Sigma Aldrich Fine Chemicals. The complete statement of the components and quantitative composition of AP135 is tabulated in table 2.

TABLE 2
AP135 Starting Materials

| Component | Mole % |
|---|---|
| 3,9-Diethylidene-2,4,8,10- | 42.9 |
| tetraoxaspiro[5.5]undecane | |
| Tri(ethylene glycol) | 38.1 |
| Tri(ethylene glycol) poly(glycolide) | [9.52:9.52] |
| [Glycolide:TEG] | |
| Anhydrous Tetrahydrofuran (THF) | N/A |

### Example 2

### Long-Acting Injectable Formulation with Buprenorphine/Polyorthoesters

Table 3 provides example buprenorphine compositions for long-acting injectable formulations. The inventive formulations were compared to BUPRENEX®, which is a commercially available formulation sold by Reckitt & Colman, Inc.

TABLE 3

| | | Route of |
|---|---|---|
| Ingredient | Dose (mg/kg) | administration |
| Buprenorphine 0.03% w/v solution | 0.01 | subcutaneous |
| BUPRENEX ® | | |
| (comparative) | | |
| Buprenorphine 0.5% w/w | 0.1 | subcutaneous |
| Polyorthoester (APF626) | | |
| Buprenorphine 1% w/w | 0.1 | subcutaneous |
| Polyorthoester (APF626R) | | |
| Buprenorphine 0.5% w/w | 0.1 | subcutaneous |
| Polyorthoester (APF579) | | |
| Buprenorphine 1% w/w | 0.1 | subcutaneous |
| Polyorthoester (APF579R) | | |
| Buprenorphine 2% w/w | 0.1 | subcutaneous |
| Polyorthoester (APF580) | | |

Long-acting buprenorphine compositions containing 0.5%, 1% and 2% w/w (buprenorphine/polyorthoester) were administered once subcutaneously to dogs at a dosage rate of 0.1 mg/kg to compare the resulting plasma levels with a single administration of BUPRENEX® 0.03% w/v solution at 0.01 mg/kg administered subcutaneously (Table 2). BUPRENEX® is administered every 6-12 hours to control pain in dogs and cats. The plasma levels obtained from administration of BUPRENEX® to about 12 hours have provided an indication of target plasma level to achieve an analgesic effect for the long-acting buprenorphine compositions.

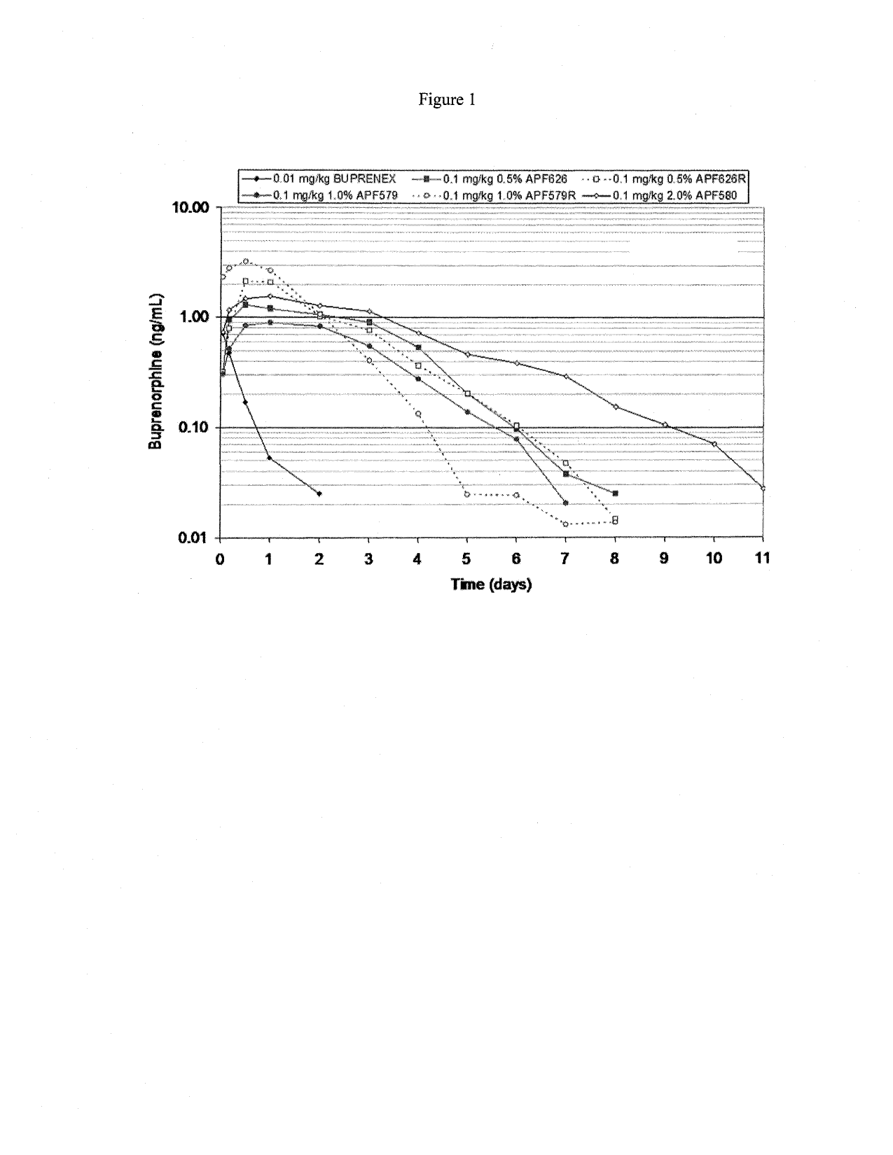

Results showed that the BUPRENEX® plasma levels in dogs was about 0.7 ng/mL at the 2 hour time point then fell below 0.2 ng/mL at the 12 hour time point, which indicated that to achieve an analgesic effect the buprenorphine in the plasma should be greater than 0.2 ng/mL (FIG. 1). As shown in FIG. 1, the buprenorphine compositions of the present invention produced longer lasting plasma levels as compared to the commercially known product BUPRENEX® and showed plasma levels supportive of an analgesic effect within 2 hours of administration, with controlled Cₘₐₓ concentrations similar to the commercial reference solution. These buprenorphine compositions administered at this dose provided plasma levels indicative of an analgesic effect for about 3 to 8 days depending on the formulation used.

### Example 3

### Long-Acting Injectable Formulation with Buprenorphine/Polyorthoesters

Table 4 provides example buprenorphine compositions for long-acting injectable formulations.

TABLE 4

| | | Route of |
|---|---|---|
| Ingredient | Dose (mg/kg) | administration |
| Buprenorphine 2% w/w Polyorthoester | 0.11 | subcutaneous |
| (APF580R) | | |
| Buprenorphine 2% w/w Polyorthoester | 0.22 | subcutaneous |
| (APF580R) | | |
| Buprenorphine 2% w/w Polyorhtoester | 0.34 | subcutaneous |
| (APF580R) | | |

Long-acting buprenorphine composition containing 2% w/w (buprenorphine/polyorthoester) was administered to 6 dogs per group at various dosage rates (0.11, 0.22, and 0.34 mg/kg) to determine plasma levels indicative of an analgesic effect (Table 3).

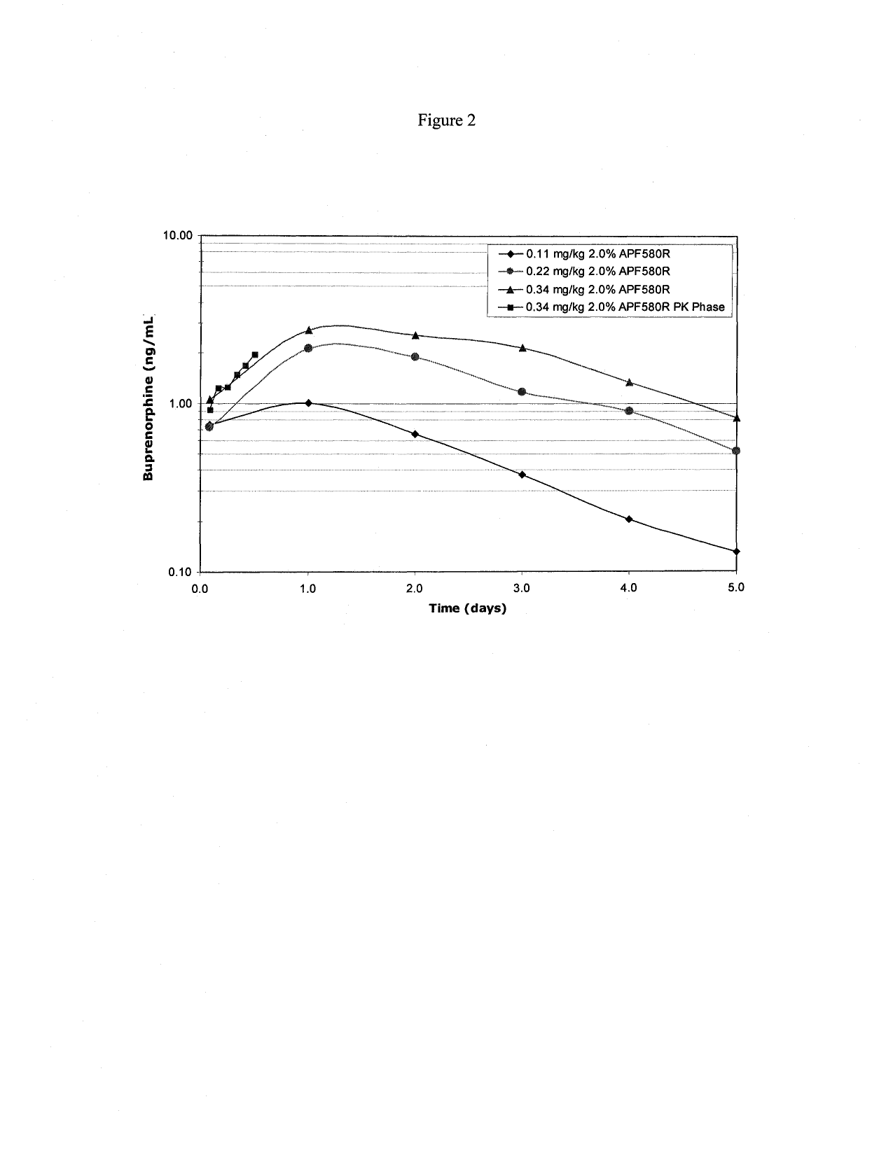

The 2% buprenorphine composition produced longer lasting plasma levels in a dose-dependant manner (FIG. 2) compared to BUPRENEX®. The PK Phase of the 2% buprenorphine composition dosed at 0.34 mg/kg showed plasma levels supportive of an analgesic effect 2 hours post-administration. The 0.11 mg/kg dose demonstrated plasma levels at or above 0.2 ng/mL through day 4, indicating four days of analgesia. The 0.22 mg/kg and 0.34 mg/kg doses demonstrated plasma levels above 0.5 ng/mL through day 5, indicating more than 5 days of analgesia.

### Example 4

### Long-Acting Injectable Formulation with Buprenorphine/Polyorthoesters

Table 5 provides example buprenorphine compositions for long-acting injectable formulations. The inventive formulations were compared to TEMGESIC®, which is a commercially available formulation sold by Schering-Plough (92 Rue Baudin, 92300 Levallois Perret, France).

TABLE 5

| | Dose | Route |
|---|---|---|
| Ingredient | (mg/kg) | of administration |
| Buprenorphine 0.03% w/v solution | 0.02 | Intravenous |
| TEMGESIC ® | | |
| (comparative) | | |
| Buprenorphine 0.5% w/w Polyorthoester | 0.025 | subcutaneous |
| (APF626R) | | |
| Buprenorphine 0.5% w/w Polyorthoester | 0.05 | subcutaneous |
| (APF626R) | | |
| Buprenorphine 0.5% w/w Polyorthoester | 0.075 | subcutaneous |
| (APF626R) | | |

Long-acting buprenorphine composition containing 0.5% w/w (buprenorphine/polyorthoester) was administered once to 4 dogs per group at various dosage rates (0.025, 0.05, and 0.075 mg/kg) subcutaneously to compare plasma levels and analgesic efficacy in a thermal threshold and nociceptive withdraw reflex model with TEMGESIC® 0.03% w/v solution at 0.01 mg/kg administered once intravenously (IV) (Table 4).

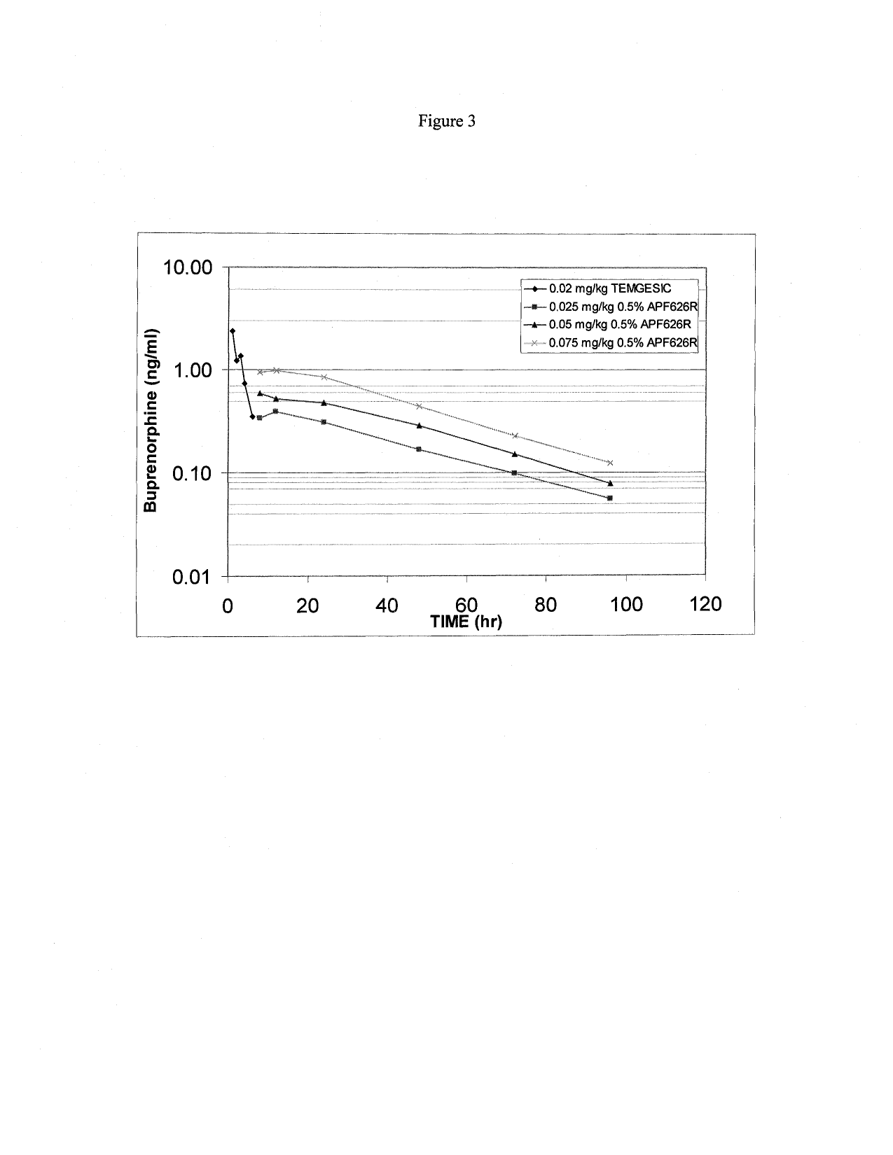

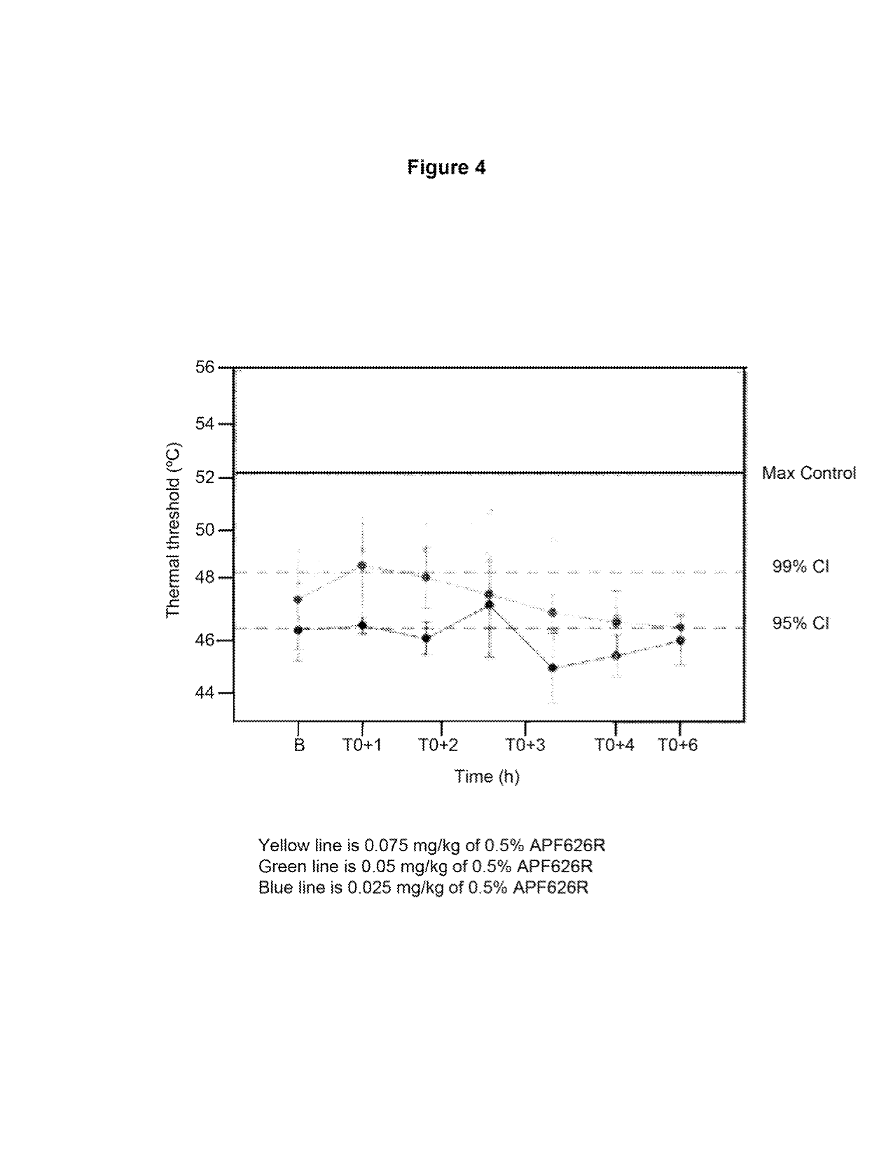

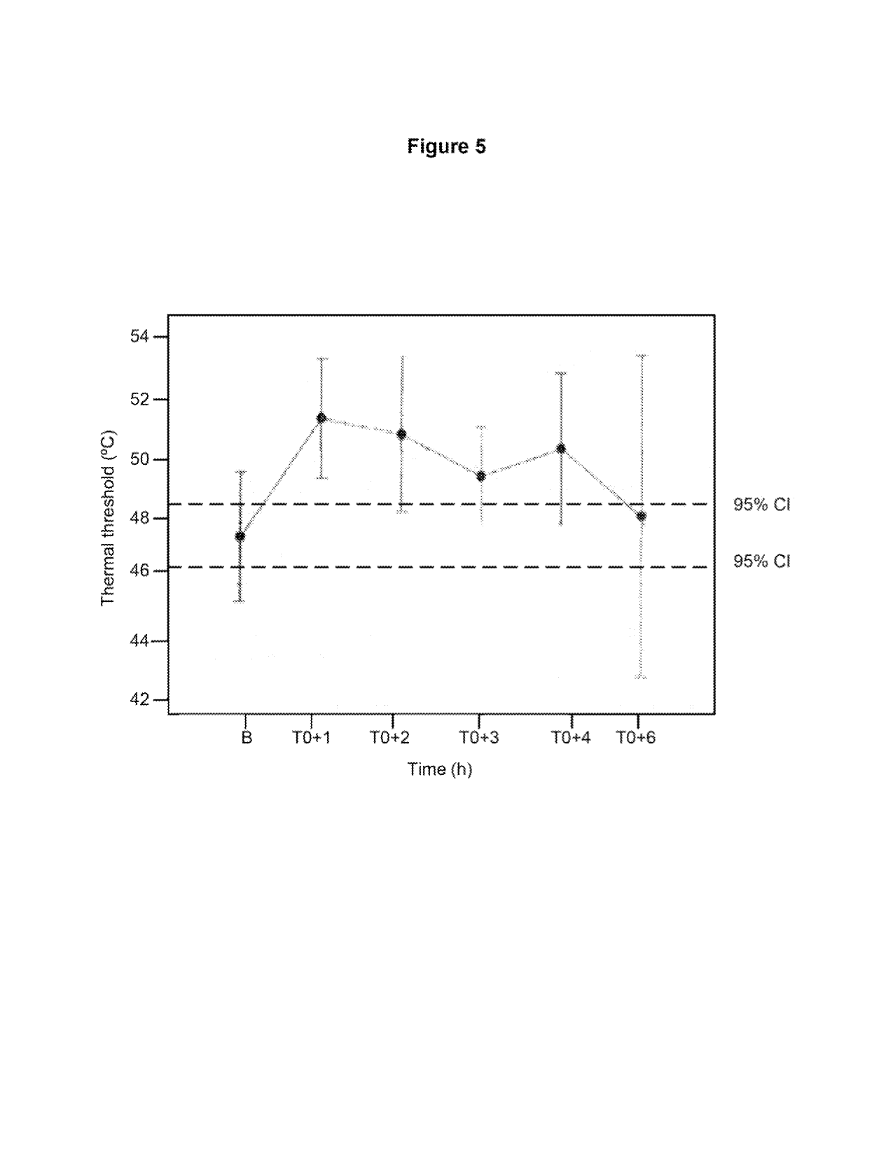

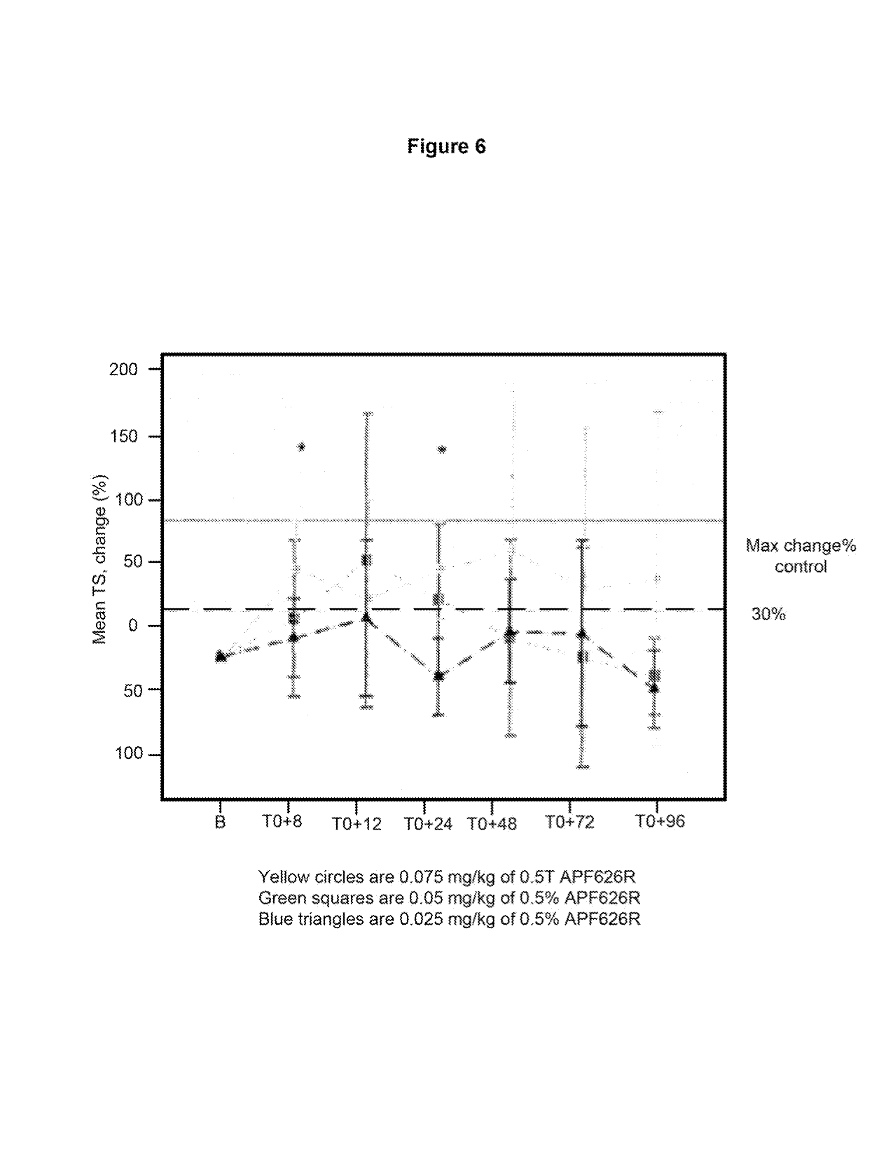

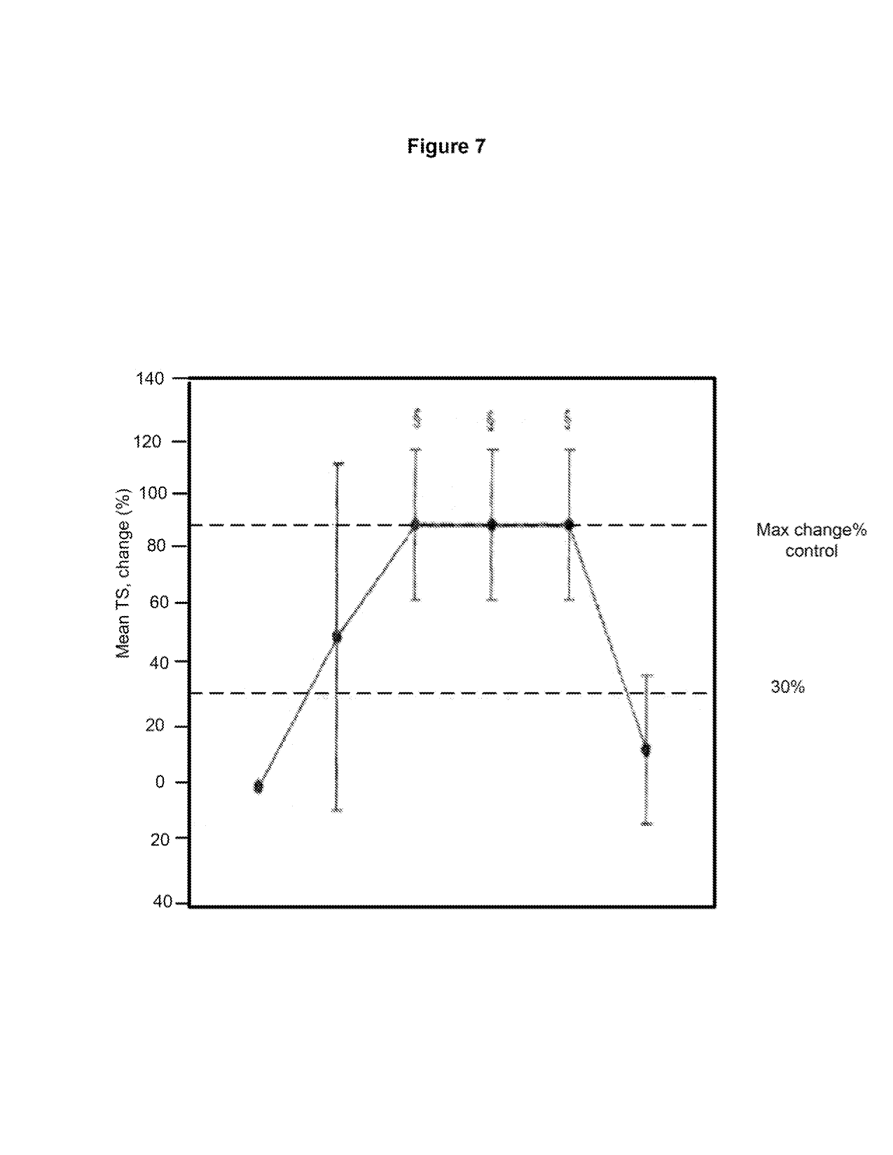

The 0.5% buprenorphine composition made according to the present invention produced longer lasting plasma levels in a dose-dependant manner (FIG. 3) than TEMGESIC®. The 0.5% buprenorphine composition at these dosages showed plasma levels for up to 4 days. Preliminary data suggests that the 0.5% buprenorphine composition showed a dose-related antinociceptive effect which correlated well with the measured plasma concentrations. Furthermore, this preliminary data suggests the 0.075 mg/kg dose has longer lasting antinociceptive activity (thermal threshold, FIGS. 4 and 5) and antihyperanlgesic efficacy (TS) at least 96 hours (FIG. 6) than TEMGESIC (FIG. 7). The preliminary results indicate that the formulations according to the present invention provide a surprising analgesic effect compared with commercially available formulations.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A long-acting injectable formulation comprising an analgesic, a polyorthoester and optionally a pharmaceutically acceptable excipient or carrier.

2-10. (canceled)
